(19) United States
(12) Patent Application Publication
(10) Pub. No.: US 2006/0051306 A1
(43) Pub. Date: Mar. 9, 2006

(54) Antiperspirant emulsion compositions

(76) Inventors: Nathan Charles Brown, Wiltshire (GB); Hugh Rieley, Bebington (GB); Ian Karl Smith, Bebington (GB); Joann Elizabeth Stockton, Bebington (GB)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(21) Appl. No.: 10/518,320

(22) PCT Filed: May 22, 2003
(86) PCT No.: PCT/EP03/05471

§ 371 Date: Aug. 17, 2005

(30) Foreign Application Priority Data
Jun. 18, 2002 (GB) 0213999.6

(51) Int. Cl. A61K 8/89; A61K 8/18

(52) U.S. Cl. 424/65

(57) ABSTRACT

A w/o emulsion antiperspirant composition comprising a dissolved antiperspirant salt, an emulsifier and, in a disperse phase separate from the dissolved antiperspirant salt, a polymer comprising Brønsted acid groups.

This invention relates to the field of antiperspirant and deodorant formulation science. More specifically, it relates to high performance antiperspirant compositions having a water-in-oil (w/o) emulsion structure.

In co-pending application PCT/EP01/13253, the applicants disclose high performance antiperspirant compositions comprising an antiperspirant salt and a polymer comprising Brønsted acid groups that acts as a co-gellant for the antiperspirant salt when mixed therewith in the presence of water, the antiperspirant salt and the polymer being physically separate from one another prior to application.

Other systems comprising an antiperspirant salt and a polymer comprising Brønsted acid groups are cited in the above co-pending application. However, none of these other systems offer the combination of physical stability and high performance delivered by the w/o emulsion compositions of the present invention.

W/o emulsion compositions have previously been employed as antiperspirant products. For example, EP 812,182 (Unilever PLC) discloses a base for an antiperspirant aerosol composition in the form of a w/o emulsion and comprising a dissolved aluminium salt, a volatile silicone, and a silicone surfactant.

We have now discovered that the performance of a w/o emulsion composition comprising a dissolved antiperspirant salt may be enhanced by the presence, in a phase separate from the dissolved antiperspirant salt, of a polymer comprising Brønsted acid groups. In addition, such systems have acceptable physical stability; a fact which is highly surprising, considering the inherent thermodynamic instability of emulsion systems.

Thus, according to a first aspect of the present invention, there is provided a w/o emulsion antiperspirant composition comprising a dissolved antiperspirant salt, an emulsifier and, in a disperse phase separate from the dissolved antiperspirant salt, a polymer comprising Brønsted acid groups.

According to a second aspect of the invention, there is provided a method of reducing perspiration comprising the application to the human body of a w/o emulsion composition comprising a dissolved antiperspirant salt and an emulsifier, and the co-application, from a disperse phase separate from the dissolved antiperspirant salt, of a polymer comprising Brønsted acid groups.

According to a third aspect of the invention, there is provided a method of manufacture of an antiperspirant composition, said method comprising emulsification of an aqueous solution of an antiperspirant salt in an oil continuous phase, followed by mixing of the emulsion so formed with a separate phase comprising a polymer comprising Brønsted acid groups.

The w/o emulsion antiperspirant compositions of the present invention comprise an antiperspirant salt dissolved in the aqueous dispersed phase. The polymer comprising Brønsted acid groups serves to enhance the performance of the antiperspirant salt when the two components come into intimate contact on application. However, it is important that intimate contact between the two components is prevented prior to their application and it is for this reason that the polymer comprising Brønsted acid groups is applied as a separate phase. Premature interaction between the two components leads to the production of a water-insoluble complex that is not an effective antiperspirant.

Premature interaction between the two components may be prevented by applying them from different compositions and this is one method of reducing perspiration according to the invention. Alternatively, a composition according to the first aspect of the invention may be applied.

In compositions according to the first aspect of the invention, it is essential that the polymer comprising Brønsted acid groups exists in a separate phase from the dissolved antiperspirant salt. This may be accomplished by suspending the polymer comprising Brønsted acid groups as a solid in an oil continuous phase. Thus, in one embodiment, there is provided a w/o emulsion antiperspirant composition comprising a dissolved antiperspirant salt and, suspended as a solid in an oil continuous phase, a polymer comprising Brønsted acid groups.

In an alternative embodiment of the first aspect of the invention, there is provided a w/o emulsion antiperspirant composition comprising a dissolved antiperspirant salt, an emulsifier, and, emulsified as a separate dispersed phase, an aqueous solution of a polymer comprising Brønsted acid groups. Compositions of this type are described as “dual” emulsions in this specification. To achieve such compositions, it is essential that the polymer is soluble in water, preferably having a solubility of 5 g/l or greater, more preferably 10 g/l or greater and most preferably 50 g/l or greater. It is also essential that the polymer solution is emulsified in an oil phase; this may be achieved using the same emulsifier as used for the emulsification of the antiperspirant solution or a different one.

The compositions of the invention comprise an oil continuous phase. The compositions may comprise silicone oil, hydrocarbon oil, and/or ester oils. When more than one oil is present, it may be preferred that the oils are miscible; although immiscibility may sometimes be desirable. In order to avoid the need for shaking of liquid compositions prior to use, it is preferred that only one oil continuous phase is present in such compositions. It is preferred that compositions of the invention comprise silicone oil and it is further preferred that the silicone oil is comprised in the oil continuous phase. Silicone oils may be cyclic or linear, examples include Dow Corning silicone fluids 344, 345, 244, 245, 246, 556, and the 200 series; Union Carbide Corporation Silicones 7207 and 7158; and General Electric silicone SF1202. Alternatively or additionally, non-silicone oils may be used; such materials include mineral oils, hydrogenated polyisobutene, polydecene, paraffins, isoparaffins of at least 10 carbon atoms, and aliphatic or aromatic ester oils (e.g. isopropyl myristate, lauryl myristate, isopropyl palmitate, diisopropyl sebecate, diisopropyl adipate, or C₈ to C₁₈ alkyl benzoates).

The compositions of the invention also comprise at least one aqueous dispersed phase. The proportion of aqueous dispersed phase(s) within the total composition (excluding any volatile propellant that may be present) is typically from 50% to 90%, particularly from 50% to 70% when used in stick compositions and particularly from 70% to 90%, especially from 75% to 85%, when used in liquid or cream/soft solid compositions, all percentages being by weight. The mean droplet size of the aqueous dispersed phase comprising the antiperspirant salt is preferably from 1 to 25 μm, in particular from 1 to 10 μm, and especially from 1 to 7 μm. When the composition also comprises an aqueous dispersed phase comprising the polymer, the mean droplet size of this dispersed phase is preferably from 1 to 25 μm, in particular from 1 to 10 μm, and especially from 5 to 7 μm. The mean droplet sizes referred to are Sauter D(4,3) means, as determined by light scattering techniques.

The Antiperspirant Salt

Antiperspirant (AP) salts for use herein are often selected from astringent salts including, in particular, aluminium, zirconium, and mixed aluminium-zirconium salts, including both inorganic salts, salts with organic anions, and complexes. Preferred antiperspirant salts are aluminium, zirconium, and aluminium-zirconium chlorides, oxychlorides, and chlorohydrates salts. Particularly preferred antiperspirant salts are polynuclear in nature, meaning that the cations of the salt are associated into groups comprising more than one metal ion.

Aluminium halohydrates are usually defined by the general formula Al₂(OH)ₓQ_{y}.wH₂0 in which Q represents chlorine, bromine or iodine, x is variable from 2 to 5 and x+y=6 while wH₂O represents a variable amount of hydration. Aluminium chlorohydrate (ACH) is an especially preferred active.

Zirconium salts are usually defined by the general formula ZrO(OH)₂₋ₓQₓ.wH₂O in which Q represents chlorine, bromine or iodine; x is from about 1 to 2; w is from about 1 to 7; and x and w may both have non-integer values. Preferred are zirconyl oxyhalides, zirconiun hydroxyhalides, and combinations thereof. Non-limiting examples of zirconium salts and processes for making them are described in Belgian Patent 825,146, Schmitz, issued Aug. 4, 1975 and U.S. Pat. No. 4,223,010 (Rubino).

AP salts as used in the invention may be present as mixtures or complexes. Suitable aluminium-zirconium complexes often comprise a compound with a carboxylate group, for example an amino acid. Examples of suitable amino acids include tryptophan, β-phenylalanine, valine, methionine, β-alanine and, most preferably, glycine.

In some embodiments, it is desirable to employ complexes of a combination of aluminium halohydrates and zirconium chlorohydrates with amino acids such as glycine, which are disclosed in U.S. Pat. No. 3,792,068 (Procter and Gamble Co.). Certain of these Al/Zr complexes are commonly called ZAG in the literature. ZAG actives generally contain aluminium, zirconium and chloride with an Al/Zr ratio in a range from 2 to 10, especially 2 to 6, an Al/Cl ratio from 2.1 to 0.9 and a variable amount of glycine. Actives of this preferred type are available from Westwood, from Summit and from Reheis.

Other actives that may be utilised include astringent titanium salts, for example those described in GB 2,299,506.

AP salts are preferably incorporated into compositions of the invention in an amount of from 0.5 to 60%, particularly from 5 to 30% or 40% and especially from 5 or 10% to 30 or 35% by weight.

The AP salt is generally dissolved in water prior to emulsification. The aqueous AP salt solution is typically of concentration from 10% to 70%, in particular from 25% to 60%, and especially from 40% to 60% by weight. The w/o emulsion formed from the AP salt solution, prior to the addition of the separate polymer phase and any volatile propellant, has a typical proportion of dispersed phase of from 50% to 90%, in particular from 50% to 70% when used in stick compositions and from 70% to 90%, especially from 75% to 85%, when used in liquid or cream/soft solid compositions, all percentages being by weight.

The Polymer

The polymers of the present invention comprise Brønsted acid groups and act as co-gellants for the AP salt when mixed therewith in the presence of water, for example water in human sweat, at a temperature of 37° C. or less. The co-gelation results in a thickened state of matter—that is to say, the three component system (polymer, AP salt, water) has a higher viscosity than that of an aqueous solution of either the polymer or AP salt alone. Without wishing to be bound by theory, it is believed that the co-gelation involves chemical interaction between the Brønsted acid groups on the polymer and hydrated metal cations of the AP salt.

A simple test may be used to determine whether or not a polymer is able to act as a co-gellant: if mixing of an aqueous solution of the polymer with an aqueous solution of the AP salt results in an increase in viscosity, then the polymer is a co-gellant for the AP salt.

In many embodiments of the invention, it is preferred that the water solubility of the polymers used, when measured at 37° C., is preferably 5 g/l or greater, more preferably 10 g/l or greater, and most preferably 50 g/l or greater. It is preferred that the polymers form true solutions in water, rather than dispersions; such true solutions typically having an absorbance of less than 0.2, preferably less than 0.1 (for a 1 cm pathlength at 600 nm) measured using a Pharmacia Biotech Ultrospec 200 Spectrophotometer or similar instrument. It is also desirable that the polymer is water soluble at pH 7; the attainment of said pH generally requiring a certain amount of neutralisation of the Brønsted acid groups present.

When the polymer is present as a suspended solid, it is preferred (particularly in liquid compositions) that the polymer is slow to dissolve in water, taking more than 8 weeks, preferably more than 16 weeks, at ambient temperature, to dissolve in the aqueous phase comprising the dissolved antiperspirant salt to an extent that causes thickening or precipitation of solid.

The Brønsted acid groups in the polymer may be present in their protonated form or may be present in their neutralised form as salt groups. Both partially-neutralised and fully-neutralised acidic polymers may be employed in the present invention. Suitable Brønsted acid groups include carboxylic acid groups, sulphonic acid groups, and phosphonic acid groups. Carboxylic acid groups are particularly preferred. Brønsted acid groups are preferably present at a concentration of greater than 0.1 mmole per gram of polymer, more preferably at a concentration of greater than 1.0 mmole/g of polymer, and most preferably at a concentration of greater than 3.0 mmole/g of polymer. Concentrations expressed of Brønsted acid groups relate to monobasic Brønsted acid groups and should be reduced pro rata for polybasic Brønsted acid groups. Latent Brønsted acid groups, such as anhydrides or other groups that generate Brønsted acid groups on addition to water, may also be present.

When the polymer is present as a suspended solid, it is preferred (particularly in liquid compositions) that the level of Brønsted acid groups in the polymer is limited to a level of less than 6 mmole/g, more preferably less than 5 mmole/g, and most preferably less than 4 mmole/g. In this way, more stable compositions result.

Preferred polymers are organic polymers, in particular, organic polymers possessing only limited positive charge, that is to say having less than 50 mole %, preferably less than 25 mole %, of positively-charged monomer units. Especially preferred organic polymers are nonionic and anionic polymers. Typical polymers possess carbon backbones, optionally interrupted by ester or amide links.

The acid value of a polymer is a widely used means of characterisation. Acid values generally express the acidity of a polymer in terms of the number of milligrams of potassium hydroxide base required to fully neutralise one gram of the polymer. Thus, the unit of measurement can be abbreviated to mg KOH/g.

Typical polymers used in the present invention have acid values of greater than 160. The polymers preferably have acid values of greater than 320, more preferably greater than 450. Especially preferred polymers have acid values greater than 580. These acid values are based on the polymer in its fully protonated state; that is to say, the actual in-use extent of neutralisation of the polymer is ignored in respect of the ‘acid value’. Acid values may be measured experimentally or may be estimated theoretically.

When using the latter method, acid anhydride groups present in a polymer should be counted as two acid groups, such latent acid groups generally being hydrolysed to di-acids by potassium hydroxide.

The preferred carboxylic acid groups may be introduced into the polymer by inclusion of monomers such as acrylic acid, methacrylic acid, maleic acid, itaconic acid, crotonic acid, maleic anhydride, or itaconyl anhydride in the polymer. When the only source of Brønsted acid groups are anhydride monomers, it is required that the anhydride groups are at least partially hydrolysed prior to use of the polymer. Polymers comprising a mixture of any of the above acid and/or anhydride monomers may also be advantageously employed. Particularly preferred polymers are those derived, at least in part, from maleic acid and/or maleic anhydride monomers.

It is sometimes desirable to include other monomers in the polymer. Suitable monomers include methyl vinyl ether, C₁-C₈ alkyl acrylates and methacrylates, vinyl acetate, ethylene, and propylene. The inclusion of such monomers may aid polymer synthesis, ease handling and/or formulation of the polymer, and may improve the performance of the polymer as a co-gellant.

The molecular weight of the polymer is preferably in the range of 500 to 5,000,000, in particular 10,000 to 3,000,000 and especially 100,000 to 2,500,000. Selection of an appropriate molecular weight for the polymer may lead to benefits in terms of ease of formulation, product aesthetics (particularly product feel), and product performance.

Particularly preferred polymers are co-polymers of methyl vinyl ether and maleic acid/anydride.

The polymer is preferably incorporated into a composition in an amount of from 0.1% to 10% by weight, more preferably from 0.5% to 5% by weight, and most preferably from 1% to 4% by weight of said composition.

When the polymer is present as a suspended solid, the particle size of the polymer is generally between 0.1 and 200 μm, preferably with a mean particle size of from 3 to 50 μm, the mean particle size being the Sauter D(4,3) mean, as determined by light scattering techniques.

When the polymer is present as an aqueous solution emulsified as a separate dispersed phase, it is preferably used as a solution of concentration from 5% to 50%, more preferably from 10% to 30%, and most preferably from 15% to 20% by weight. When a w/o emulsion is formed from the polymer salt solution, prior to mixing with the w/o AP emulsion, it typically has a proportion of dispersed phase of from 50% to 90%, in particular from 50% to 70% when used in stick compositions and from 70% to 90%, especially from 75% to 85%, when used in liquid or cream/soft solid compositions, all percentages being by weight.

The weight ratio of the AP salt to the polymer is preferably 25:1 or less, 1:10 or greater, particularly between 25:1 and 1:10, and especially between 10:1 and 1:5.

The Emulsifier

An emulsifier is an essential component of the w/o emulsion comprising the dissolved antiperspirant salt and may also be associated with the independent polymer phase. The emulsifier may be an anionic, cationic, zwitterionic, or nonionic surfactant; nonionic surfactants being preferred. The proportion of emulsifier in the total composition may be from 0.1% to 5%, preferably from 0.2% to 3.5%, more preferably from 0.25% to 2.5%, and most preferably from 0.4% to 0.6%, particularly for liquid compositions.

It is desirable to use an emulsifier or a mixture of emulsifiers with an overall HLB value in a range from 2 to 10, preferably from 3 to 8. A mixture of emulsifiers may comprise a surfactant of high HLB and a surfactant of low HLB, blended to give a suitable overall HLB.

High HLB emulsifiers include nonionic esters or ethers comprising a polyoxyalkylene moiety, especially a polyoxyethylene (POE) moiety containing from 2 to 80, and especially from 5 to 60, ethylene oxide (EO) units. Polyoxypropylene (POP) emulsifiers may also be employed, as may emulsifiers comprising one or more polyhydroxylated units such as glycerol, sorbitol, or some other alditol. The emulsifier must also comprise a hydrophobic moiety, for example an alkyl, alkenyl, or aralkyl group, normally containing from about 8 to 50 carbons and particularly from 10 to 30 carbons. The hydrophobic moiety can be either linear or branched and is often saturated, though it can be unsaturated, and it is optionally fluorinated. The hydrophobic moiety can comprise a mixture of chain lengths, for example those deriving from tallow, lard, palm oil, sunflower seed oil or soya bean oil. Examples of suitable high HLB emulsifiers include C₁₆ to C₁₈ alcohols ethoxylated with 10 to 25 ethylene oxide residues and PEG-15-25 stearate or distearate. Other suitable examples include C₁₀-C₂₀ fatty acid mono, di or tri-glycerides. Further examples include C₁₈-C₂₂ fatty alcohol ethers of polyethylene oxides with 8 to 12 EO units.

Low HLB emulsifiers, typically of HLB from 2 to 6, include fatty acid mono- or possibly di-esters of polyhydric alcohols such as glycerol, sorbitol, erythritol or trimethylolpropane. The fatty acyl moiety is often from C₁₄ to C₂₂ and is saturated in many instances, including cetyl, stearyl, arachidyl and behenyl. Examples include monoglycerides of palmitic or stearic acid, sorbitol mono or diesters of myristic, palmitic or stearic acid, and trimethylolpropane monoesters of stearic acid.

Emulsifiers that are silicone derivatives, by which it is meant emulsifiers that have a lipophilic silicone chain, are particularly preferred, especially when the continuous phase of the composition comprises silicone oil. Examples of such emulsifiers include polyoxyalkylene derivatives of dimethylpolysiloxanes, in particular POE, POP, or POE-co-POP derivatives. Such derivatives may terminate in C₁ to C₁₂ alkyl groups. Such emulsifiers may also be named dimethicone copolyol silicone surfactants, for example cetyl dimethicone copolyol.

Suitable emulsifiers and co-emulsifiers are widely available under many trade names including Abil™, Arlacel™, Brij™, Cremophor™, Dehydrol™, Dehymuls™, Emerest™, Lameform™, Pluronic™, Prisorine™, Quest PGPH™, Span™, Tween™, SF1228, DC3225C and Q2-5200.

Other Components

Other components may optionally be included in the compositions of the invention.

Structurants and emulsifiers are highly desirable in certain product forms. Structurants, when employed, are preferably present at from 1% to 30% by weight of a composition, whilst emulsifiers are preferably present at from 0.1% to 10% by weight of a composition. In roll-on compositions, such materials help control the rate at which product is dispensed by the roll ball. In stick compositions, such materials can form gels or solids from solutions or suspensions. Suitable structurants for use in such compositions include cellulosic thickeners such as hydroxypropyl cellulose and hydroxyethyl cellulose, fibre-forming structurants such as 12-hydroxystearic acid, esters of 12-hydroxystearic acid, amides of 12-hydroxystearic acid, stearic acid, behenic acid and di- and tri-glycerides thereof, N-lauroyl-glutamic acid dibutyl amide, 2-dodecyl-N,N′-dibutyl-succinamide, and dibenzylidene sorbitol. Partially or fully esterified disaccaharides, for example cellobiose octanoates, may also be used, as may structurants like dextrin palmitate or an aliphatic ester having a C₁₂-C₃₀ fatty acyl group and a C₁₂-C₂₄ fatty alcohol residue, like cetearyl behenate. Sterols (e.g. β-sitoserol) and sterol esters (e.g. oryzanol) are also suitable for use, when used in combination. Emulsion pump sprays, roll-ons, creams, and gel compositions can be formed using a range of oils, waxes, and emulsifiers. Suitable emulsifiers include steareth-2, steareth-20, steareth-21, ceteareth-20, glyceryl stearate, cetyl alcohol, cetearyl alcohol, PEG-20 stearate, and dimethicone copolyol. Suspension aerosols, roll-ons, sticks, and creams require structurants to slow sedimentation (in fluid compositions) and to give the desired product consistency to non-fluid compositions. Suitable structurants include sodium stearate, stearyl alcohol, cetyl alcohol, hydrogenated castor oil, beeswax, synthetic waxes, microcrystalline wax, paraffin waxes, candelilla wax, dibutyl lauroyl glutamide, alkyl silicone waxes, quaternium-18 bentonite, quaternium-18 hectorite, silica, and propylene carbonate. Some of the above materials also function as suspending agents in certain compositions.

A volatile propellant is an additional component used in most aerosol compositions. Volatile propellants may be used at a level of from 95% to 30%, preferably from 90% to 40% by weight. The invention is also suitable for use in low-VOC aerosol compositions comprising propellant at a level from 30% to 50% or 55% by weight. Suitable propellants include liquefied hydrocarbons or halogenated hydrocarbon gases. (particularly fluorinated hydrocarbons such as 1,1-difluoroethane and/or 1-trifluoro-2-fluoroethane) that have a boiling point of below 10° C. and especially those with a boiling point below 0° C. It is especially preferred to employ liquefied hydrocarbon gases, and especially C₃ to C₆ hydrocarbons, including propane, isopropane, butane, isobutane, pentane and isopentane and mixtures of two or more thereof.

Other propellants that may be used include alkyl ethers, such as dimethyl ether or compressed non-reactive gases such air, nitrogen or carbon dioxide.

Certain sensory modifiers are further desirable components in the compositions of the invention. Such materials are preferably used at a level of up to 20% by weight of the composition. Emollients, humectants, volatile oils, non-volatile oils, and particulate solids that impart lubricity are all suitable classes of sensory modifiers. Examples of such materials include cyclomethicone, dimethicone, dimethiconol, isopropyl myristate, isopropyl palmitate, talc, finely-divided silica (e.g. Aerosil 200), particulate polyethylene (e.g. Acumist B18), polysaccharides, corn starch, C12-C15 alcohol benzoate, PPG-3 myristyl ether, octyl dodecanol, C₇-C₁₄ isoparaffins, di-isopropyl adipate, isosorbide laurate, PPG-14 butyl ether, glycerol, hydrogenated polyisobutene, polydecene, titanium dioxide, phenyl trimethicone, dioctyl adipate, and hexamethyl disiloxane.

Fragrance is also a desirable additional component in the compositions of the invention. Suitable materials include conventional perfumes, such as perfume oils and also include so-called deo-perfumes, as described in EP 545,556 and other publications. Levels of incorporation are preferably up to 4% by weight, particularly from 0.1% to 2% by weight, and especially from 0.7% to 1.7% by weight.

It should be noted that certain components of compositions perform more than one function. Such components are particularly preferred additional ingredients, their use often saving both money and formulation space.

Further additional components that may also be included are colourants, conventional anti-microbials, and preservatives, for example C₁-C₃ alkyl parabens.

Product Form

The antiperspirant composition of the invention may take any of the forms known in the art. The composition may take the form of a stick, gel, cream, roll-on, squeeze spray, pump spray, or aerosol. Gel and cream compositions are given the collective name “soft solid” compositions, whilst roll-on, squeeze spray, pump spray, and aerosol compositions are collectively termed “liquid” compositions. Each product form contains its own selection of additional components, some essential and some optional. The types of components typical for each of the above product forms may be incorporated in the corresponding compositions of the invention.

Method of Manufacture

The method of manufacture of antiperspirant compositions according to the invention comprises emulsification of an aqueous solution of an antiperspirant salt in an oil continuous phase, followed by mixing of the emulsion so formed with a separate phase comprising a polymer comprising Brønsted acid groups. In general, the separate phase comprising the polymer comprising Brønsted acid groups is added as dispersion of the polymer in an oil continuous phase. The dispersed polymer may be in the form of solid particulates or as emulsified aqueous solution droplets. Preferably the oil continuous phase of the polymer dispersion comprises one or more oils common to the continuous phase of the antiperspirant salt emulsion to which it is added.

Dual emulsions according to the invention are manufactured by preparing independent emulsions of the polymer solution and the antiperspirant salt solution and then mixing the two. The emulsion of the antiperspirant salt solution is preferably subjected to high shear mixing, typically involving shearing at over 4000 rpm, prior to mixing with the polymer phase. This can lead to a stability benefit. Once formed, a dual emulsion composition may be used as a liquid or soft solid composition, or, with a suitable structurant present, it may be cooled to give a stick composition. For aerosol compositions, the preferred manufacturing procedure involves addition of a volatile propellant after the formation of the dual emulsion.

### EXAMPLES

The invention will now be further described by means of the following non-limiting examples.

TABLE 1
Emulsion Aerosol Compositions with Solid Polymer

| | |
|---|---|
| Product Ingredients | Level (wt %) |

| Trade Name | Chemical Name | Example 1 | Example 2 |
|---|---|---|---|
| Abil EM 90 | Cetyl dimethicone | 0.12 | 0.12 |
| emulsifier | copolyol | | |
| DC245 | Cyclomethicone | 2.78 | 2.78 |
| Eutanol G | Octyldodecanol | 0.50 | 0.50 |
| Aloxicoll L | ACH (50% solution) | 10.00 | 20.00 |
| DC1501 | D5 cyclopentasiloxane | 1.00 | 1.00 |
| | and dimethiconol | | |
| Gantrez | Poly(methyl vinyl | 1.00 | 1.50 |
| AN-119¹ | ether-co-maleic | | |
| | anhydride) | | |
| Water | Distilled water | 10.00 | — |
| CAP 40 | Butane, isobutane, | 74.60 | 74.10 |
| propellant | propane | | |

¹The Gantrez AN-119 used was partially hydrolysed, having a weight ratio of di-acid to anhydride of approximately 1:2 and a level of Brønsted acid groups of 3.7 mmole/g.

Examples 1 and 2 were prepared in the following manner. First, the oil phase components were stirred together at room temperature. Next, the Aloxicoll L (50% aqueous solution of ACH) and water were slowly added with an increasing amount of shear. To the w/o emulsion so formed, the Gantrez AN-119 polymer was added as a powder with minimal shear. Finally, the resulting base composition was transferred to an aluminium can and the liquefied propellant gas was added using standard techniques.

TABLE 2
Dual Emulsion Aerosol Compositions

| | Level (wt %) | | | |
|---|---|---|---|---|
| | Polymer | ACH | | |
| Trade Name | emulsion | emulsion | Example 3 | Example 4 |
| Abil EM 90 | 0.5 | 0.5 | 0.1 | 0.2 |
| DC245 | 10.0 | 10.0 | 2.08 | 4.16 |
| Fluid AP¹ | 5.0 | 5.0 | 1.04 | 2.08 |
| Perfume | 2.4 | 2.4 | 0.6 | 1.2 |
| Eutanol G | 2.0 | 2.0 | 0.42 | 0.84 |
| Aloxicoll | — | 80.0 | 10.0 | 20.0 |
| L(50% ACH) | | | | |
| Gantrez | 12 | — | 1.0 | 2.0 |
| S-95² | | | | |
| Water | 68.1 | 0.1 | 5.67 | 11.34 |
| CAP 40 | — | — | 79.09 | 58.18 |
| propellant | | | | |

¹PPG-14 butyl ether
²Poly(methyl vinyl ether co-maleic acid)

Dual emulsion aerosol composition Examples 3 and 4. were prepared in the following manner. First, the AP emulsion of Table 2 was formed in a manner analogous to that used in the preparation of the AP emulsion of Examples 1 and 2. Independently, the polymer emulsion of Table 2 was also formed in a conventional manner without the use of high shear. The required amounts of the two emulsions were then mixed to give the dual emulsion and the required amount of this base was transferred to aluminium cans and the liquefied propellant gas added using standard techniques.

Clinical evaluation of the antiperspirancy performance of Examples 1 and 3 showed 39% and 41% reduction in sweat production respectively, compared with 29% reduction in sweat production for a similar emulsion aerosol composition also comprising 10% Aloxicoll L (i.e. 5% ACH), but without the added polymer phase. Examples 2 and 4 showed 45% and 51% reduction in sweat production respectively, compared with 43% reduction in sweat production for a similar emulsion aerosol composition also comprising 20% Aloxicoll L (i.e. 10% ACH), but without the added polymer phase.

TABLE 3
Dual Emulsion Aerosol Composition

| | Amount | | |
|---|---|---|---|
| Ingredient | Polymer emulsion | ACH emulsion | Example 5 |
| Abil EM 90 | 0.5 | 0.5 | 0.1 |
| Finsolv TN¹ | — | 10.0 | 1.25 |
| DC245 | 10.0 | — | 0.83 |
| Fluid AP | 5.0 | 5.0 | 1.04 |
| Eutanol G | 2.0 | 2.0 | 0.42 |
| Aloxicoll L | — | 80.0 | 10.0 |
| (50% ACH) | | | |
| Gantrez S-95 | 12.0 | — | 1.0 |
| Water | 70.5 | 2.5 | 6.2 |
| CAP 40 | — | — | To 100 |

¹C₁₂-C₁₅ alkyl benzoate

Example 5 was prepared in analogous manner to Examples 3 and 4; the main difference being the use of Finsolv TN as the continuous phase of the ACH emulsion and the resulting production of a dual emulsion having a continuous phase comprising both silicone oil and ester oil.

TABLE 4
Dual Emulsion Cream Composition

| | Level (wt %) | | |
|---|---|---|---|
| | Polymer | ACH | |
| Ingredient | emulsion | emulsion | Example 6 |
| Silkflo 364NF¹ | 10.3 | 10.3 | 10.3 |
| Abil EM 90 | 1.0 | 1.0 | 1.0 |
| Aloxicoll L | — | 64.4 | 41.0 |
| (50% ACH) | | | |
| Gantrez S-95 | 11.6 | — | 4.1 |
| Glycerol | 2.1 | 2.1 | 2.1 |
| Water | To 100 | To 100 | To 100 |

¹Polydecene

Example 6 was manufactured via independent preparation of the polymer emulsion and the AP emulsion, in an analogous manner to that used for Examples 3 and 4, followed by mixing of the appropriate quantities and pouring into a soft solid dispenser pack.

TABLE 5
Dual Emulsion Stick Composition

| | Level (wt %) | | |
|---|---|---|---|
| | Polymer | ACH | |
| Ingredient | emulsion | emulsion | Example 7 |
| Kester Wax K62¹ | 12.7 | 12.7 | 12.7 |
| DC245 | 12.8 | 12.8 | 12.8 |
| Finsolv TN | 8.5 | 8.5 | 8.5 |
| Abil EM 90 | 0.5 | 0.5 | 0.5 |
| Aloxicoll L | — | 62.5 | 40.0 |
| (50% ACH) | | | |
| Gantrez S-95 | 11.25 | — | 4.05 |
| Acumist B18² | 2.0 | 2.0 | 2.0 |
| Perfume | 1.0 | 1.0 | 1.0 |
| Water | 51.25 | — | 18.45 |

¹Cetearyl behenate
²Micronised polyethylene

Example 7 was manufactured via independent preparation of the polymer emulsion and the AP emulsion, both at 85° C. with addition of the aqueous phase accompanying increasing shear to the emulsion being formed. After cooling to 75° C., the required quantities were mixed and poured into a stick barrel. Cooling and solidification gave the final product.

TABLE 6
Dual Emulsion Roll-On Composition

| | Level (%) | | |
|---|---|---|---|
| | Polymer | ACH | |
| Ingredient | emulsion | emulsion | Example 8 |
| Abil EM 90 | 0.7 | 0.7 | 0.5 |
| DC245 | 10.0 | 12.3 | 34.4 |
| Fluid AP | 5.0 | 5.0 | 3.7 |
| Eutanol G | 2.0 | 2.0 | 1.5 |
| Aloxicoll L | — | 80.0 | 44.0 |
| (50% ACH) | | | |
| Gantrez S-95 | 12.0 | — | 2.2 |
| Perfume | — | 2.3 | 1.3 |
| Water | 68.0 | — | 12.5 |

Example 8 was manufactured via independent preparation of the polymer emulsion and the AP emulsion, in analogous manner to Examples 3 and 4, followed by mixing of these emulsions with additional DC245 in the required amounts (polymer emulsion: ACH emulsion: DC245=18.3:55:26.7).

1. A w/o emulsion antiperspirant composition comprising a dissolved antiperspirant salt, an emulsifier and, in a disperse phase separate from the dissolved antiperspirant salt, a polymer comprising Brønsted acid groups.

2. An antiperspirant composition according to claim 1, wherein the polymer comprising Brønsted acid groups is suspended as a solid in an oil continuous phase.

3. An antiperspirant composition according to claim 1, comprising an aqueous solution of the polymer comprising Brønsted acid groups emulsified as a separate dispersed phase.

4. An antiperspirant composition according claim 1, comprising a silicone oil.

5. An antiperspirant composition according to claim 1, wherein the emulsifier is a silicone derivative.

6. An antiperspirant composition according to claim 1, wherein the emulsifier is present at from 0.4% to 0.6% by weight.

7. An antiperspirant composition according to claim 2, wherein the level of Brønsted acid groups in the polymer is less than 4 mmole/g.

8. An antiperspirant composition according to claim 1, wherein the proportion of aqueous dispersed phase(s) within the total composition is from 50% to 90% by weight, excluding a ny volatile propellant that may be present.

9. A method of reducing perspiration comprising the application to the human body of a w/o emulsion composition comprising a dissolved antiperspirant salt and an emulsifier, and the co-application, from a disperse phase separate from the dissolved antiperspirant salt, of a polymer comprising Brønsted acid groups.

10. A method of manufacture of an antiperspirant composition, said method comprising emulsification of an aqueous solution of an antiperspirant salt in an oil continuous phase, followed by mixing of the emulsion so formed with a separate phase comprising a polymer comprising Brønsted acid groups.
